(19) United States
(12) Patent Application Publication
(10) Pub. No.: US 2026/0060597 A1
(43) Pub. Date: Mar. 5, 2026

(54) METHOD AND SYSTEM FOR ELECTROPHYSIOLOGICAL DETERMINATION OF A BEHAVIORAL ACTIVITY

(71) Applicant: X-trodes LTD, Herzliya (IL)

(72) Inventors: Yael HANEIN, Caesarea (IL); Aaron GERSTON, Tel Aviv (IL); Ziv PEREMEN, Tel Aviv (IL)

(73) Assignee: X-trodes LTD, Herzliya (IL)

(21) Appl. No.: 19/105,831

(22) PCT Filed: Aug. 24, 2023
(86) PCT No.: PCT/IL2023/050903

§ 371 Date: Feb. 24, 2025

Related U.S. Application Data
(60) Provisional application No. 63/400,423, filed on Aug. 24, 2022.

(51) Int. Cl. A61B 5/397; A61B 5/00; A61B 5/11; A61B 5/257; A61B 5/296; A61B 5/313

(52) U.S. Cl. CPC A61B 5/397; A61B 5/1103; A61B 5/1116; A61B 5/1126; A61B 5/257; A61B 5/296; A61B 5/313; A61B 5/6832; A61B 5/7235

(57) ABSTRACT

A method comprises receiving electrical signals pertaining to electrophysiological biopotentials sensed from the skin of an individual over a plurality of identified channels, processing the electrical signals to provide for each channel a likelihood for local muscle activation, and analyzing the likelihoods based on the channel identification to determine a behavioral activity of the individual.

### RELATED APPLICATION

This application claims the benefit of priority of U.S. Provisional Patent Application No. 63/400,423 filed on Aug. 24, 2022, the contents of which are incorporated herein by reference in their entirety.

### FIELD AND BACKGROUND OF THE INVENTION

The present invention, in some embodiments thereof, relates to a electrophysiology and, more particularly, but not exclusively, to a method and system for electrophysiological determination of a behavioral activity.

Meditation, mindfulness, sports activity, and intermittent fasting are a few examples of lifestyle changes aimed at improving health and well-being. Our health is also strongly affected by what and how we eat, how we breadth, when we eat, how much we walk, how fast we walk, how well we sleep, how much we socialize, and many more lifestyle choices.

For decades, electroencephalography (EEG) has been a leading research and diagnostic tool for the noninvasive measurement of brain activity for a wide range of applications spanning psychology and cerebral functioning. EEG is extensively used in the research, diagnosis, and monitoring of many conditions, such as sleep disorders, seizure detection and anatomic locus identification, neurodegenerative diseases like Alzheimer's, and neurodevelopmental disorders like attention-deficit/hyperactivity disorder. More recently, EEG has additionally found its place in biometric identification, control of extrabodily devices, mental state monitoring, and gaming [1, 2, 3, 4].

### SUMMARY OF THE INVENTION

According to some embodiments of the invention the present invention there is provided a method of determining behavioral activity of an individual. The method comprises receiving electrical signals pertaining to electrophysiological biopotentials sensed from the skin over a plurality of identified channels; processing the electrical signals to provide for each channel a likelihood for local muscle activation; and analyzing the likelihoods based on the channel identification to determine behavioral activity of the individual.

According to some embodiments of the invention the method receives the signals from a set of electrodes placed on a skin of the individual, wherein each channel corresponds to one of the electrodes.

According to some embodiments of the invention the method receives the signals from the set of electrodes while the individual is performing an unconstrained behavioral activity.

According to some embodiments of the invention the processing comprises generating a square wave signal identifying onsets and cessations of electromyographic events.

According to some embodiments of the invention the processing comprises applying an energy tracking operator followed by a rectification operator to the signal.

According to some embodiments of the invention the analysis comprises thresholding amplitudes of the square wave signal.

According to some embodiments of the invention the analysis comprises thresholding times between bouts of the square wave signal.

According to some embodiments of the invention the method comprises filtering the electrical signals, prior to the processing, at frequencies specific to the behavioral activity.

According to some embodiments of the invention the filtering comprises applying different filters to different channels.

According to some embodiments of the invention the filtering comprises applying at least two filters to the same channel.

According to some embodiments of the invention the behavioral activity comprises at least one of chewing, swallowing, blinking, scratching, and changing posture.

According to some embodiments of the invention the method comprises further analyzing the likelihoods to distinguish between different postures of the individual while performing the same behavioral activity.

According to some embodiments of the invention the method comprises receiving additional signals indicative of an additional behavioral activity performed by the individual, wherein the additional signals are other than the electrophysiological biopotentials and the additional behavioral activity is other than the determined behavioral activity; and determining efficiency at which the additional behavioral activity is performed by the individual based on the determined behavioral activity.

According to some embodiments of the invention the additional signals comprise location data, and the method comprises determining the additional behavioral activity based on the location data.

According to an aspect of some embodiments of the present invention there is provided a system for determining behavioral activity of an individual. The system comprises: a set of electrodes adherable to a skin of an individual for sensing electrophysiological biopotentials from the skin over a plurality of identified channels, each corresponding to one of the electrodes. The system also comprises a processor in communication with the electrodes, and having a circuit configured to receive from the electrodes signals pertaining to the biopotentials, to process the signals to provide for each channel a likelihood for local muscle activation, and to analyze the likelihoods based on the channel identification to determine behavioral activity of the individual.

According to some embodiments of the invention the circuit is configured to generate a square wave signal identifying onsets and cessations of electromyographic events.

According to some embodiments of the invention the circuit is configured to apply an energy tracking operator followed by a rectification operator to the signal.

According to some embodiments of the invention the circuit is configured to apply thresholding to amplitudes of the square wave signal.

According to some embodiments of the invention the circuit is configured to apply thresholding to times between bouts of the square wave signal.

According to some embodiments of the invention the circuit is configured to filter the signals, prior to the processing, at frequencies specific to the behavioral activity.

According to some embodiments of the invention the circuit is configured to apply different filters to different channels.

According to some embodiments of the invention the circuit is configured to apply at least two filters to the same channel.

According to some embodiments of the invention the behavioral activity comprises at least one of chewing, swallowing, blinking, scratching, and changing posture.

According to some embodiments of the invention the circuit is configured to analyze the likelihoods to distinguish between different postures of the individual while performing the same behavioral activity.

According to some embodiments of the invention the processor is in communication with a sensor other than the electrode, and is configured to receive from the sensor additional signals indicative of an additional behavioral activity other than the determined behavioral activity, and to determine efficiency at which the additional behavioral activity is performed by the individual based on the determined behavioral activity.

According to some embodiments of the invention the additional signals comprise location data, and the processor is configured to determine the additional behavioral activity based on the location data.

According to some embodiments of the invention the location data comprises outdoor location data.

According to some embodiments of the invention the location data comprise indoor location data.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram of a method suitable for determining behavioral activity of an individual according to some embodiments of the present invention;

FIG. 2 is a schematic illustration of a system for determining behavioral activity of an individual, according to some embodiments of the present invention;

FIG. 3A shows images of a system used in experiments performed according to some embodiments of the present invention;

FIG. 3B shows a signal from electrodes 5-7 of the system of FIG. 3A, during calibration procedure performed according to some embodiments of the present invention;

FIG. 3C shows alpha wave presence during eyes open (orange) and eyes closed (blue) obtained in experiments performed according to some embodiments of the present invention;

FIG. 3D shows EEG-containing signal segments separated by 6 hours, used for RMS calculation according to some embodiments of the present invention;

FIG. 4A is a flowchart diagram of a procedure used to estimate a likelihood for local muscle activation, according to some embodiments of the present invention;

FIG. 4B shows an example of the application of the procedure of FIG. 4A to one channel;

FIG. 5 is a flowchart diagram of a thresholding procedure that was used for context prediction in experiments performed according to some embodiments of the present invention;

FIG. 6 is a flowchart diagram of a procedure used in experiments performed according to some embodiments of the present invention for detecting eye blinks;

FIGS. 7A and 7B show example context predictions during noisy signal segments, obtained in experiments performed according to some embodiments of the present invention;

FIG. 8 shows spectrograms highlighting activity in theta, alpha, beta, and EMG frequency ranges at each of seven channels recorded during experiments performed according to some embodiments of the present invention; and

FIG. 9 shows mean theta power, mean alpha power, mean beta power, dominant frequency within typical alpha range, and number of blinks, before, during, and after bout of automatically identified unconstrained eating, as obtained during experiments performed according to some embodiments of the present invention.

### DESCRIPTION OF SPECIFIC EMBODIMENTS OF THE INVENTION

The present invention, in some embodiments thereof, relates to a electrophysiology and, more particularly, but not exclusively, to a method and system for electrophysiological determination of a behavioral activity.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present embodiments employ a set of electrodes that are adhered to the skin of an individual for sensing electrophysiological biopotentials from the skin, and a processor that communicates with the electrodes for determining one or more behavioral activities of the individual. The determined behavioral activity is preferably an activity that is manifested by contraction of muscles, preferably skeletal muscles, below the skin nearby the locations at which the electrodes contact the skin. Optionally, but not necessarily, the behavioral activity is an activity that is controlled by the somatic nervous system.

At least part of the operations described herein can be implemented by a data processing system, e.g., a dedicated circuitry or a general purpose computer, configured for receiving electrical signals and executing the operations described below. At least part of the operations can be implemented by a cloud-computing facility at a remote location. At least part of the operations can be implemented by a central processing unit (CPU) of a mobile device, using dedicated software.

Computer programs implementing the method of the present embodiments can commonly be distributed to users by a communication network or on a distribution medium such as, but not limited to, a floppy disk, a CD-ROM, a flash memory device and a portable hard drive. From the communication network or distribution medium, the computer programs can be copied to a hard disk or a similar intermediate storage medium. The computer programs can be run by loading the code instructions either from their distribution medium or their intermediate storage medium into the execution memory of the computer, configuring the computer to act in accordance with the method of this invention. During operation, the computer can store in a memory data structures or values obtained by intermediate calculations and pull these data structures or values for use in subsequent operation. All these operations are well-known to those skilled in the art of computer systems.

Processing operations described herein may be performed by means of processer circuit, such as a DSP, microcontroller, FPGA, ASIC, etc., or any other conventional and/or dedicated computing system.

The method of the present embodiments can be embodied in many forms. For example, it can be embodied in on a tangible medium such as a computer for performing the method operations. It can be embodied on a computer readable medium, comprising computer readable instructions for carrying out the method operations. In can also be embodied in electronic device having digital computer capabilities arranged to run the computer program on the tangible medium or execute the instruction on a computer readable medium.

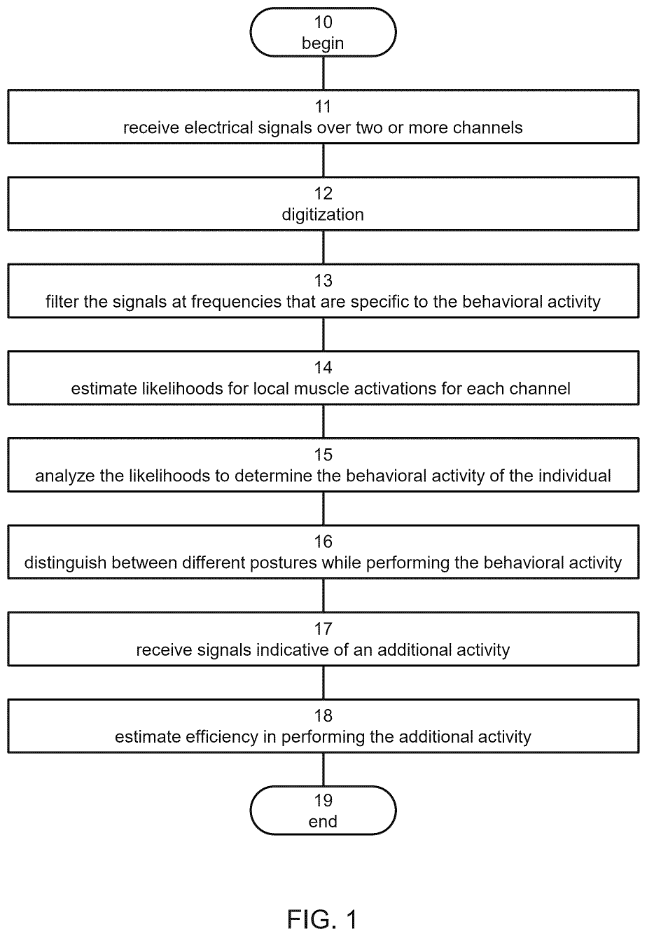

Referring now to the drawings, FIG. 1 is a flowchart diagram of a method suitable for determining behavioral activity of an individual according to various exemplary embodiments of the present invention. It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

The method of the present embodiments is preferably executed for determining one or more behavioral activities of interest. Representative examples of behavioral activities of interest that can be determined by the method include, without limitation, at least one of, more preferably at least two of: chewing, swallowing, blinking, scratching, and posture changing (e.g., rolling, standing up after sitting, sitting after lying, bending, straightening up after bending, stretching, and the like).

The method begins at 10 and optionally and preferably continues to 11 at which electrical signals are received from a set of electrodes placed on a skin of the individual, over a plurality of channels, each corresponding to one of the electrodes. The signals pertain to electrophysiological biopotentials sensed from the skin by the electrodes, and are preferably received from the electrodes while the individual is performing an unconstrained behavioral activity. In these embodiments the method preferably receives the signal without instructing the individual to perform the behavioral activity, and without receiving any synchronization signal that is indicative of the onset of the behavioral activity, other than the signals that pertain to electrophysiological biopotentials.

When the signals are received directly from the electrodes, they are typically in an analogue form, and the method preferably proceeds to 12 at which the signals are discretized. Alternatively, a discretized form of the signals can be obtained from an external source (e.g., read from a computer readable storage medium, or downloaded over a communication network from a cloud storage facility), in which case 11 and 12 can be skipped.

Whether or not the signals are discretized by the method of received in their discretized form, the discretization of the signals is preferable at least 1000 samples per second, more preferably at least 2000 samples per second, more preferably at least 3000 samples per second, for example, about 4000 samples per second or more.

Whether or not the signals are discretized by the method of received in their discretized form, each channel of the signals forms a one-dimensional array of values representing biopotentials picked up by one of the electrodes.

The method optionally and preferably proceeds to 13 at which the signals are filtered. The inventors found that use of different filtering schemes allows determining different behavioral activities. Thus, according to some embodiments of the present invention the filtering scheme and/or the cutoff frequency (or set of cutoff frequencies) of the filtering scheme are specific to the behavioral activity of interest. In a preferred embodiment, the method employs a library of behavioral activities. Each entry in the library stores a behavioral activity and a filtering scheme and/or cutoff frequencies that is/are associated with that behavioral activity. Such a library can be stored digitally, for example, in a memory of a processor that executes the method, and/or in a computer readable medium accessible by the processor. The processor can access the memory or medium, search the library for a library entry in which the behavioral activity of the library matches the behavioral activity of interest, and use the filtering schemes and/or cutoff frequency (or set frequencies) that are stored in the found library entry in order to execute operation 13.

The filtering employed at 13 can apply the same filter for all channels. Alternatively, different filters (either filters employing different schemes or filters employing the same scheme but different cutoff frequencies) are applied to different channels. The advantage of these embodiments is that it allows the method to determine behavioral activity in cases in which the behavioral activity generates different biopotential patterns at the vicinity of different electrodes.

In some embodiments of the present invention two or more different filters (either filters employing different schemes or filters employing the same scheme but different cutoff frequencies) are applied to the same channel. Also contemplated, are embodiments in which a digital channel is duplicated and different filters are applied to different copies of the same channel, thus generating two different filtered channels from the same digital channel. For example, a particular digital channel can be duplicated wherein a first copy of the digital channel is filtered with, e.g., a 50 Hz comb filter and a 30-350 Hz bandpass filter, and a second copy of the digital channel can be filtered with a 0.3-30 Hz bandpass filter, but without applying a comb filter. This exemplified procedure provides two separate digital channels that correspond to the biopotentials picked up by the same electrode, where the first copy detects muscular activity and the second channel detects ocular activity.

The advantage of using more than one filter for the same channel is that it allows the method to simultaneously determine two or more behavioral activities using signals received from the same set of electrodes.

The method proceeds to 14 at which the electrical signals are processed to estimate, for each channel, a likelihood for local muscle activation. In some embodiments of the present invention the processing 14 comprises generating, for each channel, a square wave signal, which identifies onsets and cessations of electromyographic events. The square wave signal is preferably generated solely based on the signals that signals pertain to the electrophysiological biopotentials, without using any additional synchronization signal that indicates onsets and cessations of the behavioral activity of interest. The square wave signal is generated in a manner that its instantaneous amplitudes express local muscle activation likelihoods for the respective channel.

The generation of square wave signal optionally and preferably includes application of an energy tracking operator, which is preferably a three point operator that calculates the instantaneous energy of the signal at a give time, using the amplitudes of the signal at three time points within an interval that encompasses the give time. For example, consider a signal that is sampled at n time points t₁, t₂, . . . , tₙ, to form a digitized signal represented by a one-dimensional vector of amplitudes S(tᵢ), where i=1, 2, . . . n. In this case, the energy tracking operator can calculate the instantaneous energy of the signal time tᵢ, using the amplitudes at times tᵢ₋₁, tᵢ, tᵢ₊₁.

Three point energy tracking operators suitable for the present embodiments including, without limitation, the Teager-Kaiser Energy (TKE) operator, and the Girolami-Vakman operator. For example, when the TKE operator is employed, the instantaneous energy TKE at time tᵢ can be calculated by subtracting the multiplication of the signal amplitudes at times tᵢ₋₁, and tᵢ₊₁ from the square of the signal amplitude at time tᵢ.

In some embodiments of the present invention the application of energy tracking operator is followed by application of a rectification operator to signal. Any type of rectification operator can be used, including, without limitation, an absolute value operator, an even power operator, and the like. For example, the rectification operator can square the output of the energy tracking operator. Additional operators that are contemplated for generating the square wave signal, including, without limitation, smoothing (e.g., using a moving-mean operator), erosion, and dilation.

Once the likelihoods for local muscle activation are estimated (and optionally and preferably expressed as the square wave signal), the method optionally and preferably proceeds to 15 at which the likelihoods are analyzed, based on the channel identification, to determine behavioral activity of the individual. In some embodiments of the present invention, the analysis can be based on thresholding. The thresholding can be in the amplitude domain and/or the temporal domain. The domain and thresholds are specific to the behavioral activity of interest. The present embodiments thus contemplate a library of behavioral activities, wherein each entry in the library stores a behavioral activity and a thresholding scheme (typically in the form of amplitude and/or temporal thresholds) that is specific to that behavioral activity. In various exemplary embodiments of the invention, for a given behavioral activity of interest, the signals of at least two different channels are analyzed according to at least two different thresholding schemes, respectively. In these embodiments, each entry in the library of behavioral activities includes two or more stored thresholding schemes, one thresholding scheme for each signal channel.

The library can be stored digitally, for example, in a memory of a processor that executes the method, and/or in a computer readable medium accessible by the processor. The present embodiments contemplate more than one way to utilize the library. In some embodiments, the processor accesses the memory or medium, searches the library for a library entry in which the behavioral activity of the library matches the behavioral activity of interest, and applies thresholding according to the scheme or schemes that is/are stored in the found library entry to the likelihoods estimated at 14. Depending on the result of the thresholding operation, the method can determine whether or not the individual is performing the behavioral activity of interest, or to estimate the likelihood that the individual is performing the behavioral activity of interest. In some embodiments, the processor accesses the memory or medium, loops through the entries of the library, and, for each entry of the loop, applies to the likelihoods estimated at 14 thresholding according to the scheme or schemes that is/are stored in that entry. Depending on the result of the thresholding operation, the method can determine whether or not the individual is performing a behavioral activity that is associated with the respective library entry, or to estimate the likelihood that the individual is performing the behavioral activity that is associated with the respective library entry.

The analysis 15 can alternatively or additionally comprise applying a trained machine learning procedure to the likelihoods of the various channels. In these embodiments, the input to the machine learning procedure is the likelihoods of the channels (for example, the square wave signal of obtained for each channel), and the output of the machine learning procedure is the behavioral activity of the individual that best matches the input likelihoods. In some embodiments, the input to the machine learning procedure includes the discretized signals before or after filtering, and in some embodiments of the present invention the input to the machine learning procedure includes features extracted from the signals or signals obtained by other manipulations.

Representative examples of machine learning procedures suitable for the present embodiments, include, without limitation, clustering, association rule algorithms, feature evaluation algorithms, subset selection algorithms, support vector machines, classification rules, cost-sensitive classifiers, vote algorithms, stacking algorithms, Bayesian networks, decision trees, neural networks, instance-based algorithms, linear modeling algorithms, k-nearest neighbors (KNN) analysis, ensemble learning algorithms, probabilistic models, graphical models, logistic regression methods (including multinomial logistic regression methods), gradient ascent methods, singular value decomposition methods and principle component analysis.

In some embodiments of the present invention the method proceeds to 16 at which the likelihoods are analyzed also for distinguishing between different postures of the individual while performing the same behavioral activity. Such a distinguishing can be done by thresholding and/or by machine learning. When thresholding is employed, each entry in the library of behavioral activities includes, in addition to the above information, also a posture that corresponds to the thresholding scheme(s) for the behavioral activity of the entry. In these embodiments, at least one of the behavioral activities of the library includes two or more entries, for respective two or more different posture. The processor can then access the memory or medium that stores the library, and utilize the library as further detailed hereinabove. Thus, in some embodiments, the processor determines whether or not, or estimates the likelihood that, the individual is performing the behavioral activity of interest, and also determines, or estimates, the posture of the individual while performing the behavioral activity of interest; and in some embodiments, the processor loops through the entries of the library, and determines, or estimates the likelihood that, the individual is performing a behavioral activity that is associated with the respective library entry, and also determines, or estimates, the posture of the individual while performing the behavioral activity that is associated with the respective library entry.

In some embodiments of the present invention the method continues to 17 at which additional signals indicative of another activity are received. The other activity is optionally and preferably also a behavioral activity, as further detailed hereinabove. The additional signals are other than the electrophysiological biopotentials received at 11. For example, the additional signals can comprise location data describing the location of the individual, in which case the other activity is determined based on the location of the individual. The location can be an outdoor location or an indoor location. For outdoor location, the additional signals can indicate whether the individual is in an outdoor urban area or an outdoor non-urban area or some specific outdoor urban area (e.g., busy street, side street, plaza), or some specific non-urban area (e.g., park, forest, beach). For indoor location, the additional signals can indicate whether the individual is in an office, a store, or at home, or the like, and can indicate a specific location within an indoor environment, e.g., in the kitchen or the living room or the study, or near a computer. Based on the location data, the method cab determine what type of additional activity the individual is involved with (e.g., working, doing chores, shopping, vacating, in transit between locations, etc.)

The method can them proceed to 18 at which the efficiency at which the individual performs the additional activity is estimated based on the behavioral activity determined at 15. As a representative example, consider a case in which the signals received at 17 are processed and it is determined that the individual is working by operating a computer (e.g., based on location data indicating that the individual is near a computer), and the method determines at 15 that the individual is talking. In this case, the method can estimate that it is likely that the efficiency of the individual's work is reduced. As another example, consider a case in which it is determined that the individual is working by operating a computer, and the method determines at 15 that the individual is chewing. In this case, the method can determine the rate of chewing and estimate the efficiency of the individual's work based on the rate of chewing (e.g., high chewing rate, which may be caused by stress, reduce the efficiency, while low chewing rate, which may indicate higher concentration state, increase the efficiency.

The method ends at 19.

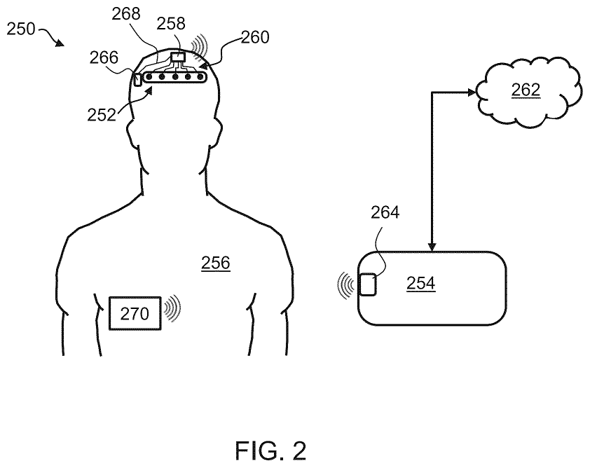

FIG. 2 is a schematic illustration of a system 250 for determining behavioral activity of an individual, according to some embodiments of the present invention. Shown are a set 252 of electrodes adherable to the skin of a subject 256. Electrodes sense electrophysiological biopotentials from the skin over a plurality of identified channels 260, each corresponding to one of electrodes 252. Electrodes 252 are optionally and preferably in the formed of a patch which can be plugged into a miniature wireless data acquisition circuit 258 that optionally and preferably amplifies and/or digitizes the signals. Circuit 258 is preferably configured to transmit the signal using a wireless transmission protocol, such as, but not limited to, Bluetooth, Z-wave, ZigBee, WIFI and the like. Also contemplated are embodiments in which circuit 258 is configured to utilize other communication technologies for transmitting the signals, including, without limitation, the Internet, GPRS, GSM, CDMA, 3G, 4G, and 5G communication technologies.

System 250 also comprises a processor 254 that optionally and preferably comprises a communication circuit 264 that communicates with the electrodes 252, preferably via data acquisition circuit 258, via one of the aforementioned protocols and technologies. Processor 254 can include dedicated circuitry, or be general purpose computer. Processor 254 can, in some embodiments of the present invention, be a CPU of a mobile device on which dedicated software is installed. Processor 254 can be a cloud-computing facility at a remote location. Combinations of these embodiments are also contemplated. For example, a CPU of a mobile device can communicates with the electrodes to receive the signals, and then transmit the signals over a communication network 262 to a remote location at which a cloud computing facility or a computing server (not shown) is located.

In any event, processor 254 receives the signals (either directly from circuit 258, or, when processor is remote, via another circuit, e.g., a local processor), processes the signals to provide for each channel a likelihood for local muscle activation, and analyzes the likelihoods based on the channel identification to determine behavioral activity of the individual. Preferably, processor 254 executes at least some of the operations described above with respect to the flowchart diagram of FIG. 1.

System 250 optionally and preferably one or more accelerometers 266 for collecting acceleration data, describing motion changes of subject 256. In these embodiments, the acceleration data collected by accelerometer 266 is also transmitted to processor 254 as one or more channels 268 that are separated from channels 260. Processor 254 can receive the acceleration data process the data to obtain information pertaining to motion changes of subject 256, and supplement that analysis of the signals from electrodes 252 with the obtained information. In some embodiments of the present invention processor 254 uses the obtained information to vary the likelihoods that are calculated using the signals from the electrodes. For example, when the processor determines the likelihood of a particular behavioral activity that is typically associated with a motion change, and the acceleration data is also indicative of such a motion change, the processor can increase the likelihood for that particular behavioral activity.

In some embodiments of the present invention processor 254 is in communication with a sensor 270 other than electrodes 252. Sensor 270 can be part of system 250 or be a component of another system, such as, but not limited to, a handheld device (e.g., a smartphone or a tablet), or a wearable device (e.g., a smartwatch, or a wearable GPS tracker), or the like. FIG. 2 illustrates a preferred embodiment in which sensor 270 is mounted on the body of individual 256, but this need not necessarily be the case, since, for some applications, it may not be necessary for sensor 270 to be mounted on the body. The communication between sensor 270 and processor 254 can be according to any technique or protocol described above with respect to circuit 258.

Processor 254 receives from sensor 270 additional signals that are indicative of an additional activity performed by the individual as further detailed hereinabove. Processor 254 processes these additional signals and determines the efficiency at which the additional activity is performed by the individual based on the behavioral activity that is determined using the electrophysiological biopotentials from electrodes 252.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Monitoring Freely Behaving Humans

The Examples below describe an algorithmic approach for analyzing electrophysiological recordings during daily and night activity of freely behaving humans. The Examples below describe the implementation of this approach along with an electrophysiological measurement device for EEG recordings during normal day and night activity. The Examples utilize a fully wireless system including a soft and dry electrode array and a miniaturized data acquisition system that enables continuous monitoring of electrophysiological signals over durations of at least 10 hours. Provided are the characteristics of the system as well as results of simultaneous recordings of cerebral, ocular, and muscular biopotentials produced by the system during instructed activity and during free behavior. Also provided is a description of a case study in which EEG activity is analyzed before and after specific behaviors, both under controlled conditions in which the behavior is constrained, and under spontaneous, freely behaving conditions in which the behaviors are unconstrained.

### Introduction

Meditation, mindfulness, sports activity, intermittent fasting are a few examples of lifestyle changes aimed at improving health and well-being. Our health is also strongly affected by what and how we eat, how we breadth, when we eat, how much we walk, how fast we walk, how well we sleep, how much we socialize, and many more lifestyle choices.

Existing technologies to monitor humans in their daily lives focus on few specific modalities such heart rate, gait, brain activity (EEG). The inventors found that they provide only a snapshot of the relevant information needed to document people's daily lifestyle choices.

The following describes an algorithmic approach to derive a wide range of parameters to describe human behavior from wearable sensors such as EEG, EMG, ECG, and accelerometers. These parameters include, without limitation, eating, breathing, sleep quality, meditation, walking (accelerometer, EMG), breathing (accelerometer), how fast a person is chewing, eating while sitting or standing, medication adherence and many more. The following Examples describe a unique combination of EEG, EMG, EOG and accelerometers.

For decades, electroencephalography (EEG) has been a leading research and diagnostic tool for the noninvasive measurement of brain activity for a wide range of applications spanning psychology and cerebral functioning. EEG is useful in research, diagnosis, and monitoring of sleep disorders, seizure detection and anatomic locus identification, neurodegenerative diseases like Alzheimer's, and neurodevelopmental disorders like attention-deficit/hyperactivity disorder. More recently, EEG has additionally found its place in biometric identification, control of extrabodily devices, mental state monitoring, and gaming [Ahn, Lee, Choi, & June 2014; Padfield, Jabalza, Zhao, Masero, & Ren, 2019; Abdulkader, Atia, & Mostafa, 2015; Rashid, et al., 2020].

The Inventors realized that the use of EEG remains largely limited to highly constrained conditions (by subject mobility, subject behavior, stimulus type, recording location, recording duration, etc.) [Makeig, Gramann, Jung, Sejnowski, & Poizner, 2009; Usakli, 2010; Gramann, et al., 2011; Picton, et al., 2000]. The Inventors found that these limitations are due several characteristics of EEG systems: physical limitations of traditional wired setups, a lack of behavioral context capturable by the body's electrophysiological output at traditional EEG electrode locations, myriad artifacts (transient disturbances in the signal) introduced by mechanical disruptions of the body-electrode interface and concurrent physiological process like heartbeat and blinking that tend to interlope on EEG signals, and application complexity (electrode location knowledge, gel application) that typically necessitates professional oversight [Sweeney, Ward, & McLoone, 2012; Reis, Hebenstreit, Gabsteiger, Von Tscharner, & Lochmann, 2014].

The present embodiments provide a solution that enjoys increased mobility and subject freedom, and is therefore useful in real-world situations, and be utilized by untrained home users. The system described herein reduces monetary and labor cost, and drives insight into authentic cognition in real-world scenarios. The system enjoys advantageous features such as portability (wireless, compact), recording stability over long periods of time (sustained power source, steadfast electrode-body interface), ability to discern behavioral context of the user (additional streams of information), and ease-of-use (dry electrodes, few components, simple user interface).

### System Characteristics

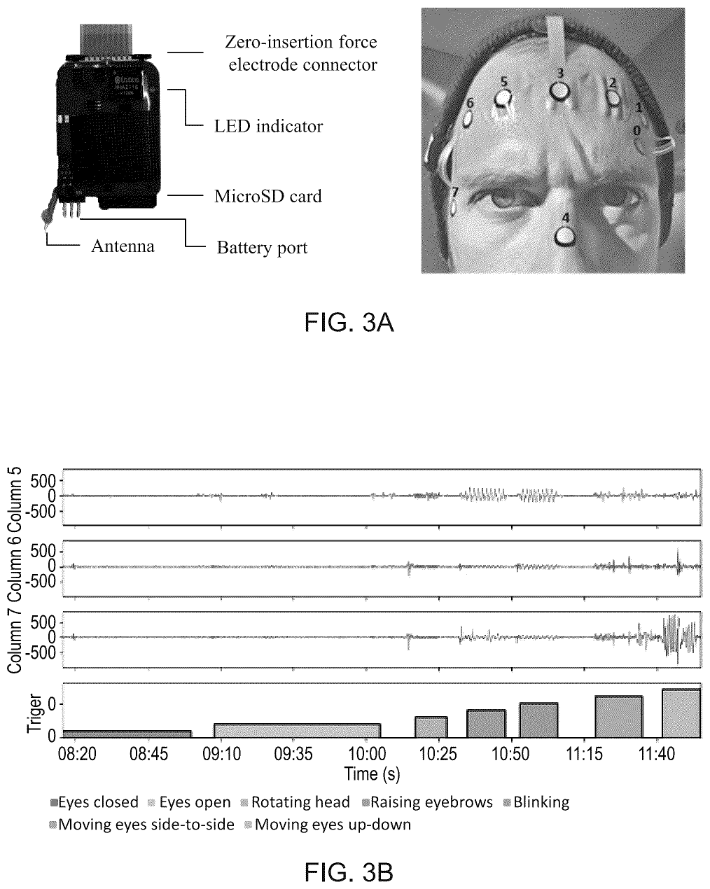

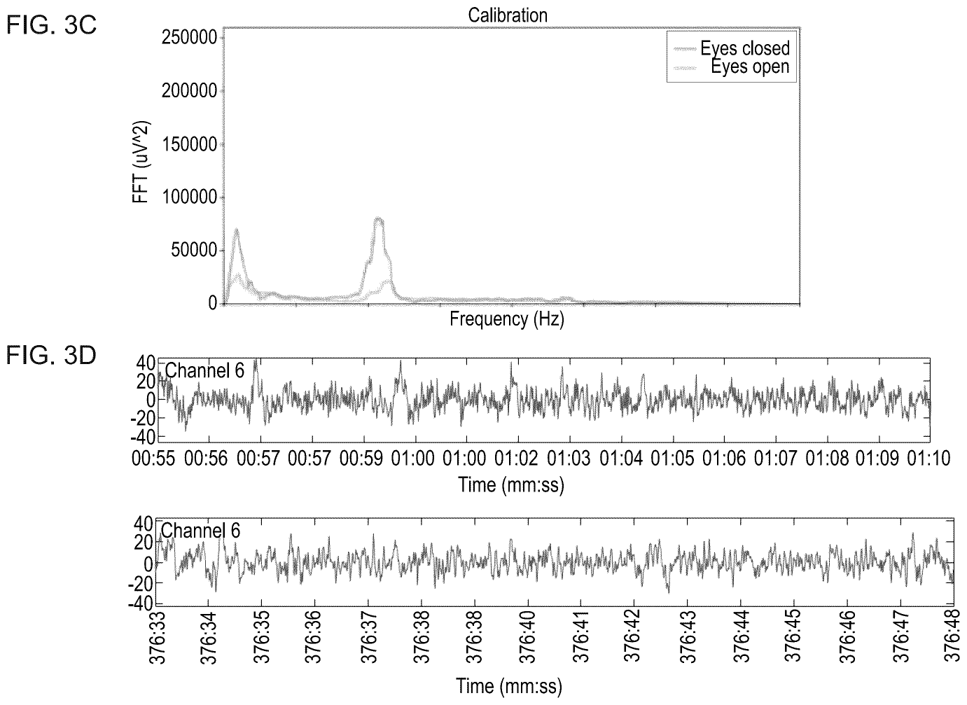

The data acquisition system used in this Example is shown in FIG. 3A. The system consisted an 8-channel unipolar electrode array screen-printed onto a flexible, adhesive polyurethane patch, and a data acquisition unit circuit that amplifies, filters and digitally stores the analog bioelectrical signals for offline analysis. Two versions of the acquisition unit circuit have been used, the specifications of each are summarized in Tables 1 and 2, below.

TABLE 1

| Max. # input | 16 | | Max SD card storage | 64 | GB |
|---|---|---|---|---|---|
| channels | | | | | |
| Sampling rate | 4000 | samples/sec | Recording limit | 12 | hours |
| | | | (620 mAh battery) | | |
| Resolution | 16 bits, 0.412 μV | | Operating voltage | 4.2 V-3.6 V | |
| | | | range | | |
| Input range | ±6 | mV | Typical current | 50 | mA |
| | | | consumption | | |
| Input capacitance | 15 | pF | # TTL sync inputs | 4 | |
| Input impedance | 1.3 GΩ @ 10 Hz | | Max sync rate | 10 | events/sec |
| Noise RMS | 2 | μV | Board size | 30 × 20 | mm |
| Board mass (without | 3.12 | gr | | | |
| batter) | | | | | |

TABLE 2

| Max. # input | 16 | | SD card storage | 8-32 | GB |
|---|---|---|---|---|---|
| channels | | | | | |
| Sampling rate | 4000 | samples/sec | Battery, duration | 100 mAh, 2.5 hours | |
| | | | | 700 mAh, 17 hours | |
| Resolution | 16 | bits | Operating voltage | 3.6-4.2 | V |
| | | | range | | |
| Input range | ±12.5 | mV | Typical current | 40 | mA |
| | | | consumption | | |

| | | | |
|---|---|---|---|
| Input impedance | 10 MΩ ∥ 6 pF | Dimensions (with | 36.8 × 40.3 × 14.8 mm |

100 mAh battery)

| | | | | |
|---|---|---|---|---|
| Noise RMS | 2 | μV | Dimensions (with | 42 × 42 × 18.5 mm |

700 mAh battery)

| | | | |
|---|---|---|---|
| Bandwidth | 0.5-700 Hz | Weight | 17 g |

(100 mAh battery)
28 g
(500 mAh battery)

The systems use in this Example were able to produce lab-quality signals outside of the lab, to maintain stability over hours of use, to be comfortable over hours of use, to be easy and simple enough to use without previous experience or expertise, and to yield a wide range of data.

To validate these characteristics, multiple several-hour-long recordings were performed using the XTR-RF-1 DAU and an electrode array designed for EEG acquisition (pictured in FIG. 3A).

### Materials

Surface electrophysiological measurements were recorded using 8-electrode arrays screen-printed on a flexible, adhesive substrate that conforms to the contours of the face. As shown in FIG. 3A, five of the 4-mm diameter silver electrodes were placed horizontally across the forehead, one on the bridge of the nose, one above one eye, and the other externally beside the other eye. A commercial 15 mm×20 mm adhesive ground electrode (Spes Medica, DENIS01526) was placed at the left mastoid. The ground electrode cable was fixed to the headband with surgical tape to prevent artifacts related to cable movement.

The data acquisition circuit was situated above the forehead. To mitigate movement artifacts and disconnections between the data acquisition circuit and the attached electrode array, the DAU was fixed to a headband or cloth hat by hook fasteners built into its underside. Data was sampled at 4000 samples/second.

Video was simultaneously recorded using an HP Omen built-in front-facing camera and OBS Studio recording software (www(dot)obsproject(dot)com/) as a safeguard to verify timing of behaviors and accuracy of automated action predictions.

### Experimental Design

Data was continuously recorded from one participant throughout the workday (up to 7 hours). Three such recordings were performed and analyzed over the span of several workdays. Each recording began with one calibration session, after which the participant carried on normally with his workday. The purpose of the calibration session was to establish a repertoire of baseline signals corresponding to a number of anticipated behaviors relevant to artifact assessment, EEG analysis during freely behaving times, and additional analyses.

Calibration consisted of the following behaviors, each performed for approximately 60 seconds each:
(a) Relaxing while looking forward at a fixed location with eyes open, without moving
(b) Relaxing while looking forward at a fixed location with eyes closed, without moving
(c) Rotating the head horizontally about the z axis
(d) Blinking periodically while looking forward at a fixed location
(e) Raising eyebrows
(f) Scrunching eyebrows
(g) Periodically moving eyes side-to-side (shifting gaze left to right)
(h) Periodically moving eyes up-down (shifting gaze top to bottom)
(i) Swallowing
(j) Chewing (a banana or granola bar)
(k) Walking naturally around the room
(1) Adjusting glasses on face

Actions performed during the freely behaving portion of the recordings included:
(i) Programming/data analysis

### (ii) Eating

(iii) Drinking coffee/tea/water
(iv) Studying/reading
(v) Socializing/chatting
(vi) Silent meditation

### Analysis

### Assessment of Signal Quality and Stability

To assess signal quality and stability, two analyses were performed: baseline signal root mean square (RMS) and alpha power.

Signal RMS is a mathematically simple indicator of overall power in the signal; in clean EEG (barring the presence of external noise contributions to the signal), values are expected to vary on the scale of several microvolts to tens of microvolts, depending on the cognitive state of the subject.

Typical EEG signals from healthy individuals reflect overlapping cerebral processes that occur primarily at frequencies between 0.5-70 Hz. One of these components, known as the alpha rhythm, roughly inhabits the 7-14 Hz band of the EEG frequency spectrum (but may slightly vary across people and possibly mental states [Berry, Brooks, Marcus, & Vaughn, 2015; Cohen, 2021)]. In typical resting-state EEG of a healthy individual, the alpha rhythm is noticeable during wakeful relaxation while eyes are closed, and is attenuated when eyes are open, or the subject is mentally occupied or asleep. When present, it can be seen visually in the signal time series as a continuously oscillating waveform or as a peak in the power spectrum.

RMS was calculated on 15-second-long quiet signal segments separated by several hours and alpha spectral power during two data segments representing intentional relaxation with eyes open and the second with eyes closed. Alpha analyses were performed on data filtered with a 50 Hz notch filter and 0.5-35 Hz 4th-order Butterworth bandpass filter. RMS calculations were performed separately on both raw and filtered data. As a measure of consistency, RMS was additionally calculated on a second data set recorded during sleep with the version described in Table 2, above.

### Multimodality

To account for common behaviors of a subject in an office environment, biopotentials corresponding to facial muscle activation and ocular activity were extracted and analyzed in parallel from the same data set. Ocular activity, including eye movements and blinks, was derived from electrodes 0 and 7 (above and externally beside the eyes, refer to FIG. 3A). After filtering the data with a 0.5-35 Hz 4th-order Butterworth bandpass filter and 50 Hz comb filter, ocular activity manifests as transient slow-wave peaks or dips in the signal in the amplitude range of hundreds of microvolts. Using a 30-400 Hz 4th-order Butterworth bandpass filter and a 50 Hz comb filter, muscle activation was derived separately in each of the 8 channels.

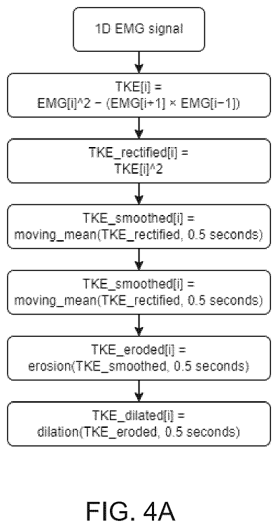

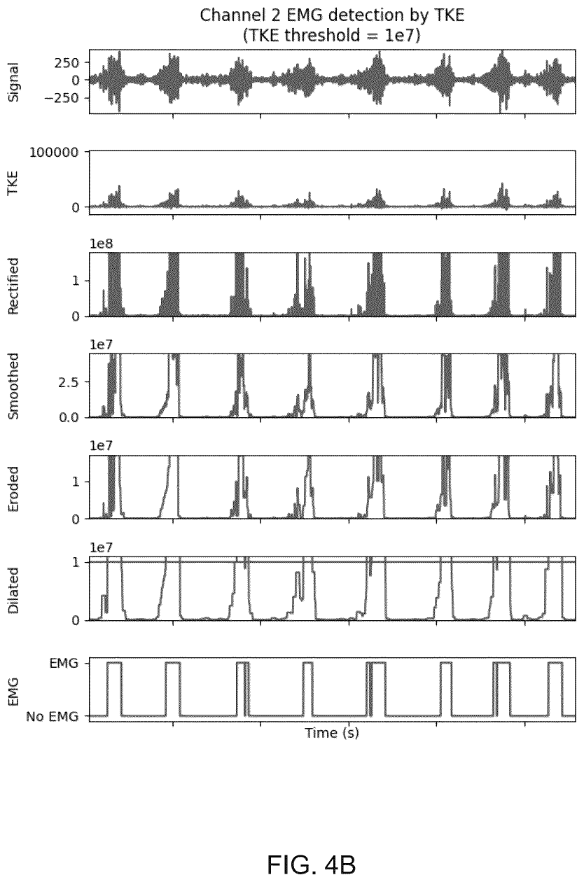

Moments of muscle contraction were derived in each channel according to the procedure described in the flowchart diagram of FIG. 4A. An example of the application of the procedure to one channel (channel 2, in the present example), using a TKE threshold of 1E7, is shown in FIG. 4B. In the exemplified procedure, TKE is calculated, squared, smoothed with a moving mean filter, eroded, and dilated before applying any thresholding. Erosion and dilation are performed with the same convolution filters to preserve EMG activation duration. The final plot in FIG. 4B illustrates a simple example of EMG activation recognition by thresholding the dilated TKE signal. A pseudo-code of the procedure is provided in Table 3, below.

TABLE 3

```
procedure detect_emg( ):
  input (array) signal_filtered
  input (numeric) sample_rate
  for i = 0 to length(signal_filtered) do:
    if i == 0 or i == length(signal_filtered) then:
      tke[i] = signal_filtered[i]
    else:
      tke[i] = signal_filtered[i]\hat{ }2 − signal_filtered[i+1] *
  signal_filtered[i+1]
  tke_rectified = tke\hat{ }2
  tke_smoothed = moving_average_filter(tke_rectified,
sample_rate/2)
  the_eroded = minimum_filter(tke_smoothed, sample_rate/2)
  tke_dilated = maximum_filter(tke_eroded, sample_rate)
  output tke_dilated
end
```

The output of this procedure is an array of muscular activation plausibility equal in length to the input signal, where a larger number simultaneously reflects both a higher likelihood of muscle activation and a stronger muscle contraction for a given data sample.

### Inferring Behavioral Context

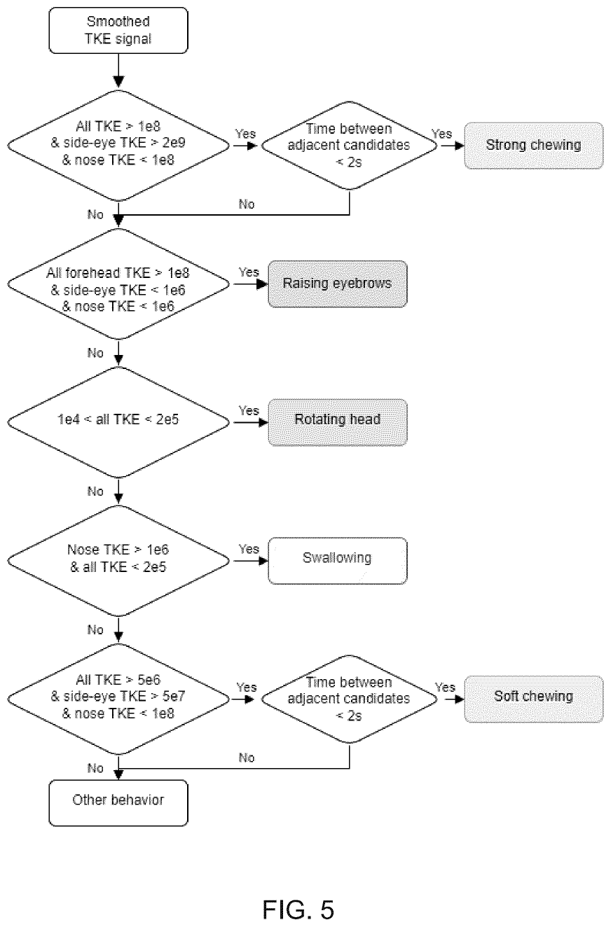

Using the calibration tasks described above, a mapping was created associating muscle activation patterns in various electrode channel combinations to respective actions. In this Example, behavioral context predictions were made using a simple logic tree based on EMG thresholds and contraction repetition timing. A representative example of a thresholding procedure that was used for context prediction is illustrated in the flowchart diagram of FIG. 5.

A pseudo-code of the procedure is provided in Table 4, below.

TABLE 4

```
procedure predict_context( ):
  input (array of arrays) tke # output of detect_emg( )
  for i = 0 to length(tke) do:
    if (tke[i, all forehead channels] > 1e8) ...
    and (tke [i, side eye channel] < 1e6) ...
    and (tke [i, nose channel] < 1e6) then:
      prediction[i] = ‘Raising eyebrows'
    if (tke[i, side eye channel] > 2e9) ...
    and (tke[i, nose channel] < 2e9) ...
    and (tke[i, all channels] > 1e8) ...
    and (time between bouts < 2 seconds) then:
      prediction[i] = ‘Strong chewing’
    if (tke[i, side eye channel] > 5e7) ...
    and (tke[i, nose channel] < 1e8) ...
    and (tke[i, all channels] > 5e6) ...
    and (time between bouts < 2 seconds) then:
      prediction[i] = ‘Soft chewing’
    else if 1e4 < tke[i, all channels] < 2e5 then:
      prediction[i] = ‘Rotating head’
    else if (tke[i, nose channel] > 1e6) ...
    and (tke[i, forehead channels] < 2e6), then:
      prediction = ‘Swallowing’
  output prediction
end
```

It is appreciated that the thresholding shown in FIG. 5 and Table 4, is not to be considered as limiting.

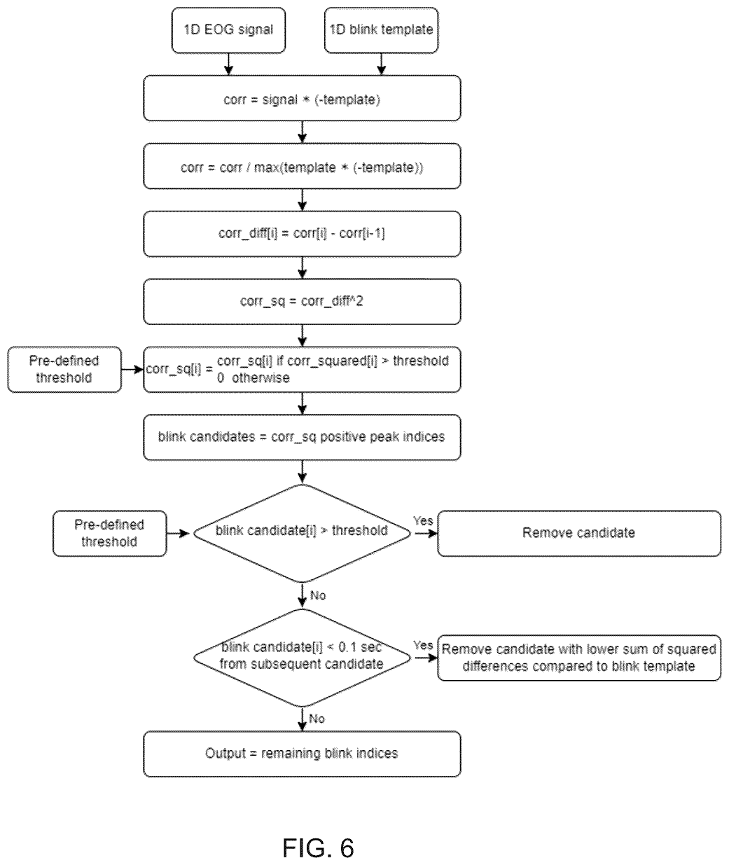

Additionally, eye blinks were identified from the ocular activity captured from the electrode located externally beside the eye. Blinks were identified via cross-correlation of the filtered side-eye signal with an established blink template. In this Example, a blink template was created using a data from a brief blinking calibration session by averaging known blink-containing signal segments in the time domain into a single 0.6-second-long blink template. Blink midpoints were located from the calibration segment as signal peaks in the side-eye channel at least 0.05 seconds long and 150 microvolts in amplitude. A flowchart diagram of a procedure used for detecting eye blinks from the entire recording's side-eye channel is illustrated in FIG. 6. A pseudo-code of the procedure is provided in Table 5, below.

TABLE 5

```
procedure find_blinks( ):
  input (array) blink_template, signal_filtered
  input (numeric) corr_threshold
  corr = convolve(signal_filtered, blink_template)
  corr_normalized = corr / max(convolve(blink_template,
blink_template)
  corr_diff = [corr[i] − corr[i−1] for i = 1 to length(corr)]
  corr_diff[corr_diff < 0] = 0
  corr_squared = corr\hat{ }2
  above_corr_threshold = corr_squared[corr_squared >
corr_threshold]
  peaks = peaks of above_corr_threshold
  blink_indices = [ ]
  for peak in peaks do:
    if peak is longer than 0.1 seconds in duration ...
    and peak is less than blink_threshold in amplitude, then:
      append peak index to blink_indices
  for candidate1, candidate2 in adjacent blink_indices < 0.1
seconds apart, do:
    SSD1 = sum of squared differences of candidate1 to
blink_template
    SSD2 = sum of squared differences of candidate2 to
blink_template
    if SSD1 > SSD2, then:
      remove candidate1 from blink_indices
    else:
      remove candidate2 from blink_indices
  output blink_indices
end
```

Blinks are often treated as pesky artifacts that infiltrate EEG signals and are thus immediately removed through techniques like ICA [Jung, Humphries, Makeig, Mckeown, & Iragui, 1998]. The inventors found that blinks can be a valuable indicator of cognition, fatigue, and myriad additional mental states (Paprocki & Lenskiy, 2017). Blink analysis (type, frequency, etc.) can therefore strengthen EEG research by providing a simultaneous supplementary window into the subject's cognition. One such analysis is showcased below, where blink frequency is used alongside common EEG features to describe cognitive effects of unconstrained behaviors before subsequent removal from EEG channel data.

### Exemplified Behavioral Activity: Chewing

The activity and blink recognition techniques described above were used to assess the cognitive effects of chewing. Myriad studies have been published describing various cognitive changes associated with mastication, most of which emphasize the before-after effects of chewing [Sakamoto, Nakata, & Kakigi, 2009].

Once bouts of chewing were located according to the procedure described in FIG. 5, theta (4-8 Hz), alpha (8-14 Hz) and beta (14-30 Hz) power, dominant frequency within the alpha band, and blink frequency were calculated in 30-second segments immediately before and after chewing. Segments containing minimal EMG leakage were selected for EEG analysis.

### Results

### Signal Quality and Stability Assessment

FIGS. 3A-D depict the main features of the system used in this Example, where FIG. 3A shows images of a system, FIG. 3B shows a signal from electrodes 5-7, FIG. 3C shows alpha wave presence during eyes open (orange) and eyes closed (blue), and FIG. 3D shows EEG-containing signal segments separated by 6 hours, used for RMS calculation. In FIG. 3D, the segments are 15 seconds long.

As demonstrated in FIG. 3B, signal amplitude can vary significantly, from several microvolts to several millivolts, according to the behavior of the subject. While the subject is still, brain waves dominate the signal recorded in forehead electrodes. Conversely, certain large movements like walking and whole-head movements tend to introduce artifacts. Strategically placed electrodes, such as those positioned above and beside the eyes and on the nose in FIG. 3A, function to isolate certain sources for subsequent artifact removal or supplementary features.

FIGS. 3C and 3D demonstrate examples of the system's signal quality and stability. FIG. 3C demonstrates that alpha activity dominated the signal spectrum during eyes closed compared to during eyes open. Average RMS calculated from the segments shown in FIG. 3D (0:55-1:10 and 6:16:33-6:16:48) were 72.62 and 45.09 μV, respectively, in the raw data and 12.04 and 10.05 μV after applying the EEG filters described above.

To further substantiate the consistency of the system, the same RMS calculation was performed again on a second data set recorded from the version described in Table 2 using a similar electrode array during sleep. RMS from segments 1:56:00-1:56:15 and 9:17:30-9:17:45 were 42.78 and 42.70 μV, respectively, in the raw data and 3.11 and 4.23 μV after applying the EEG filters described above.

### Multimodality

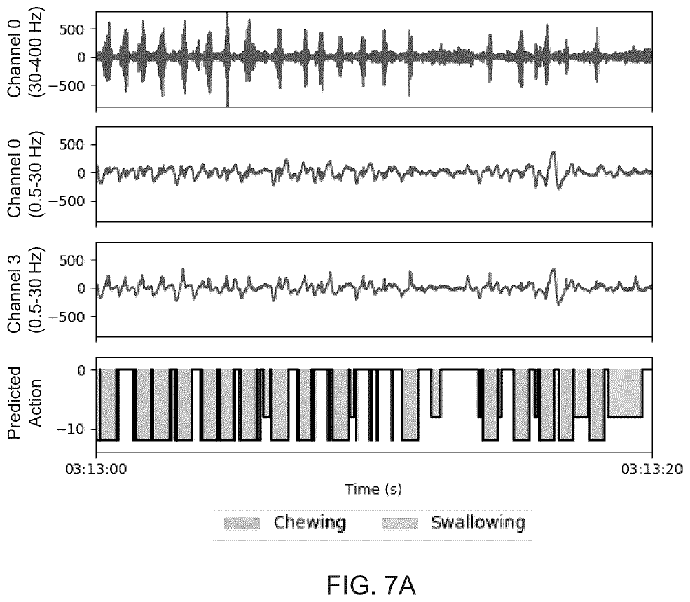

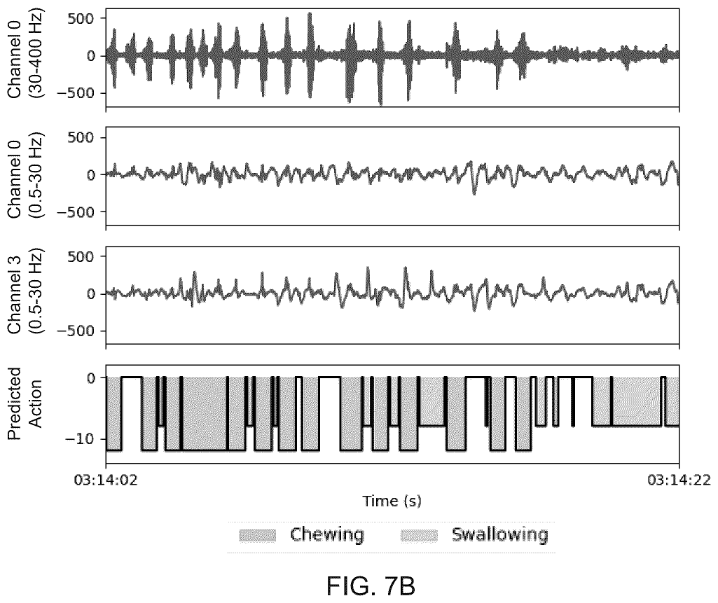

FIGS. 7A and 7B show example context predictions during noisy signal segments. FIG. 7A shows results obtained during instructed chewing: the subject was looking forward while eating either a banana or granola bar. FIG. 7B shows results obtained from unconstrained activity: the subject was eating lunch freely, without instruction. In each of FIGS. 7A and 7B, the top graph shows channel 0 (beside the eye) with a 50 Hz comb filter and 30-400 Hz bandpass filter, the middle graphs show channel 0 and channel 3 (forehead) with 50 Hz notch and 0.5-35 Hz bandpass filters, and the bottommost graph shows the predicted actions (orange=chewing, grey=swallowing), determined according to the thresholding illustrated in FIG. 5 and described in Table 4.

FIGS. 7A and 7B demonstrate the utility of strategically placed electrodes in combination with the high sampling rate to simultaneously capture several types of biopotentials from the same electrodes. By filtering the signal to retain components in the frequency range of typical muscle activity while attenuating other sources (FIGS. 7A and 7B, top row), the two actions (chewing and swallowing in the present example) together indicate the behavior of the subject during the given time frame: eating, as verified post-hoc via video and manual annotations created during the recording. Concurrent filtering of the same data set designed to retain components that resonate at frequencies typical of cerebral and ocular activity reveal entirely different signal morphologies (middle graphs of FIGS. 7A and 7B).

### Cognitive Analysis of Unconstrained Behavior

Signal components from strong muscular, ocular, or certain other activities may leak into the filtered signals despite EEG-optimized spectral filters. This phenomenon was exploited by the Inventors, wherein EMG-based action predictions were used to automatically identify presence of artifacts occluding EEG signals while at the same time providing behavioral context.

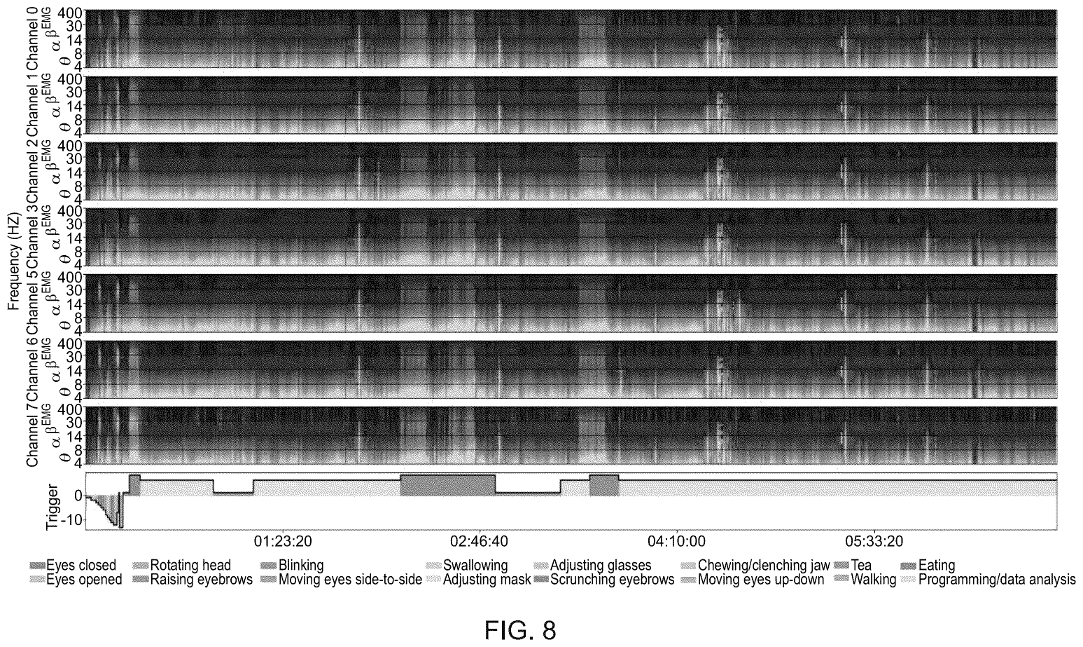

FIG. 8 shows spectrograms highlighting activity in theta, alpha, beta, and EMG frequency ranges at each of the seven channels throughout one entire recording. The bottom graph shows the actual behaviors performed.

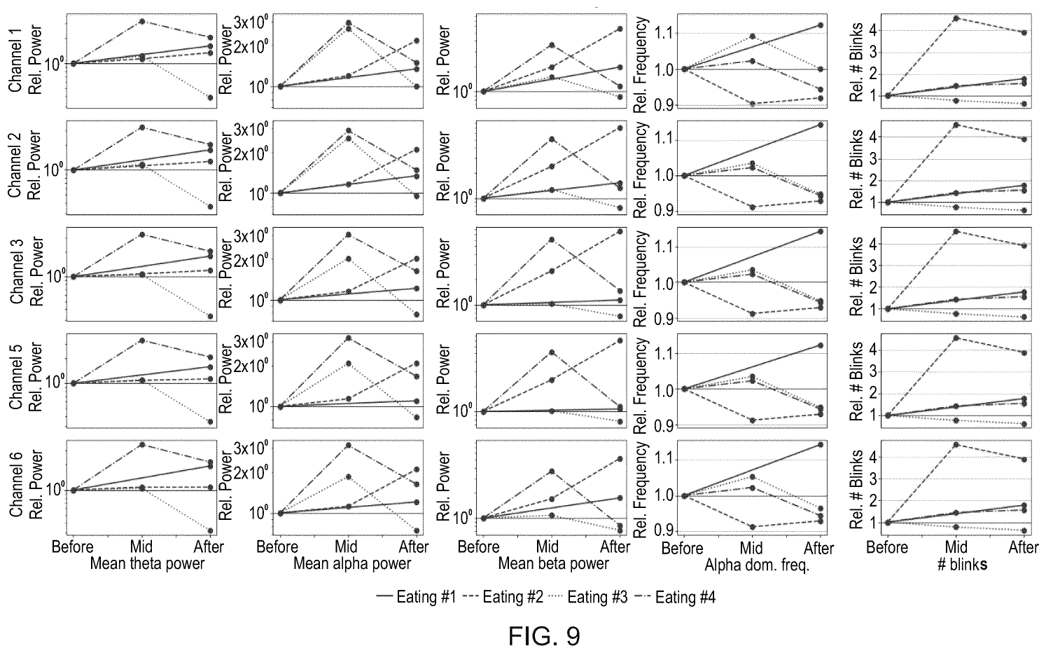

After identifying signal segments relevant to the behavior of interest (eating, in the present example), visual inspection of the signal and its spectrogram (FIG. 8), allowed locating nearby signal segments that appear to contain relatively clean EEG. Such signal segments occurring immediately before and after the behavior of interest (eating, in the present example) were analyzed and are presented in FIG. 9.

FIG. 9 shows, for each of channels 1 through 6, the mean theta power (left column), mean alpha power (second column from left), mean beta power (third column from left), dominant frequency within typical alpha range (fourth column from left), and number of blinks (right column), before, during, and after bout of automatically identified unconstrained eating.

As shown in FIG. 9, blinking, as well as alpha, beta, and theta power were all greater after eating than before eating in three out of four occurrences. Alpha dominant frequency, conversely, was lower after eating than before eating in three out of four occurrences.

The findings presented above introduce insight into the cognitive effects of chewing under natural, unconstrained circumstances. Previous studies, by contrast, were performed with specific foodstuffs ingested under constrained and unnatural conditions that may have themselves altered the cognitive effect under study.

The above examples demonstrate the ability of the system of the present embodiments to detect behavioral activity. The system exhibits recently low baseline noise and stability of signal quality over many hours. Leveraging the flexibility of the system, it is able to simultaneously record multiple biopotential signals without the need for additional electrodes or sensors. These unique features facilitate unprecedented possibility to record EEG in natural settings while guided by automatic behavior recognition, using a single, compact, wireless device.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

[1] M. Ahn, M. Lee, J. Choi and S. C. Jun, “A Review of Brain-Computer Interface Games and an Opinion Survey from Researchers, Developers and Users,” Sensors (Basel), vol. 14, no. 8, pp. 14601-33, 2014.
[2] N. Padfield, J. Jabalza, H. Zhao, V. Masero and J. Ren, “EEG-Based Brain-Computer Interfaces Using Motor-Imagery: Techniques and Challenges,” Sensors (Basel), vol. 19, no. 6, p. 1423, 2019.
[3] S. N. Abdulkader, A. Atia and M.-S. M. Mostafa, “Brain computer interfacing: Applications and challenges,” Egyptian Informatics Journal, vol. 16, pp. 213-30, 2015.
[4] M. Rashid, N. Sulaiman, A. P. Abdul Majeed, R. M. Musa, A. F. Abu Nasir, B. S. Bari and S. Khatun, “Current Status, Challenges, and Possible Solutions of EEG-Based Brain-Computer Interface: A Comprehensive Review,” Frontiers in Neurorobotics, p. 14:25, 2020.
[5] S. Makeig, K. Gramann, T.-P. Jung, T. J. Sejnowski and H. Poizner, “Linking brain, mind and behavior,” International Journal of Psychophysiology, vol. 73, pp. 95-100, 2009.
[6] A. B. Usakli, “Improvement of EEG Signal Acquisition: An Electrical Aspect for State of the Art of Front End,” Computational Intelligence and Neuroscience, vol. 2010, 2010.
[7] K. Gramann, J. T. Gwin, D. P. Ferris, K. Oie, T.-P. Jung, C.-T. Lin, L.-D. Liao and S. Makeig, “Cognition in action: imaging brain/body dynamics,” Reviews in the Neurosciences, vol. 22, no. 6, pp. 593-608, 2011.
[8] T. Picton, S. Bentin, P. Berg, E. Donchin, S. Hillyard, R. Johnson, Jr., G. Miller, W. Ritter, D. Ruchkin, M. Rugg and M. Taylor, “Guidelines for using human event-related potentialsto study cognition: Recording standardsand publication criteria,” Psychophysiology, vol. 37, pp. 127-52, 2000.
[9] K. T. Sweeney, T. E. Ward and S. F. McLoone, “Artifact Removal in Physiological Signals—Practices and Possibilities,” IEEE TRANSACTIONS ON INFORMATION TECHNOLOGY IN BIOMEDICINE, vol. 16, no. 3, pp. 488-500, 2012.
[10] P. M. Reis, F. Hebenstreit, F. Gabsteiger, V. Von Tscharner and M. Lochmann, “Methodological aspects of EEG and body dynamics measurements during motion,” Frontiers in Human Neuroscience, vol. 8, p. 156, 2014.
[11] R. Berry, R. Brooks, C. Marcus and B. Vaughn, The AASM Manual for the Scoring of Sleep and Associated Events, 2.2 ed., Darien, Illinois: American Academy of Sleep Medicine, 2015.
[12] M. Cohen, “A data-driven method to identify frequency boundaries in multichannel electrophysiology data,” J Neurosci Methods, vol. 347, p. 108949, 2021.
[13] T. Jung, C. Humphries, S. Makeig, M. Mckeown and V. Iragui, “Extended ICA Removes Artifacts from Electroencephalographic Recordings,” Advances in Neural Information Processing Systems, vol. 10, pp. 983-900, 1998.
[14] R. Paprocki and A. Lenskiy, “What does eye-blink rate variability dynamics tell us about cognitive performance?,” Front Hum Neurosci., vol. 11, p. 620, 2017.
[15] K. Sakamoto, H. Nakata and R. Kakigi, “The effect of mastication on human cognitive processing: A study using event-related potentials,” Clinical Neurophysiology, vol. 120, pp. 41-50, 2009.
[16] T. Yagyu, I. Kondakor, K. Kochi, T. Koenig, D. Lehmann, T. Kinoshita, T. Hirota and T. Yagyu, “Smell and taste of chewing gum affect frequency domain eeg source localizations,” International Journal of Neuroscience, vol. 93, no. 3-4, pp. 205-16, 1998.
[17] A. P. Allen, T. J. Jacob and A. P. Smith, “Effects and after-effects of chewing gum on vigilance, heart rate, EEG and mood,” Physiology & Behavior, vol. 133, pp. 244-51, 2014.
[18] Y. Masumoto, T. Morinushi and M. Takigawa, “Spectral analysis of changes in electroencephalographic activity after the chewing of gum,” Psychiatry and Clinical Neurosciences, vol. 52, no. 6, pp. 587-92, 1998.
[19] C. Karson, K. Berman, E. Donnelly, W. Mendelson, J. Kleinman and R. Wyatt, “Speaking, thinking, and blinking.,” Psychiatry research, vol. 5, no. 3, pp. 243-6, 1981.
[20] K. Kotowski, K. Stapor, J. Leski and M. Kotas, “Validation of Emotiv EPOC+ for extracting ERP correlates of emotional face processing,” Biocybernetics and Biomedical Engineering, vol. 38, no. 4, pp. 773-81, 2018.
[21] P. Sawangjai, S. Hompoonsup, P. Leelaarporn, S. Kongwudhikunakorn and T. Wilaiprasitporn, “Consumer grade EEG Measuring Sensors as Research Tools: A Review,” IEEE Sensors Journal, vol. 14, p. 553352, 2015.
[22] W. D. Hairston, K. W. Whitaker, A. J. Ries, J. M. Vettel, J. C. Bradford, S. E. Kerick and K. McDowell, “Usability of four commercially-oriented EEG systems,” Journal of Neural Engineering, vol. 11, no. 4, 2014.
[23] O. E. Krigolson, C. C. Williams, A. Norton, C. D. Hassall and F. L. Colino, “Choosing MUSE: Validation of a Low-Cost, Portable EEG System for ERP Research,” Frontiers in Neuroscience, vol. 11, p. 109, 2017.
[24] E. Ratti, S. Waninger, C. Berka, G. Ruffini and A. Verma, “Comparison of Medical and Consumer Wireless EEG Systems for Use in Clinical Trials,” Frontiers in Human Neuroscience, vol. 11, p. 398, 2017.
[25] T. Radüntz, “Signal Quality Evaluation of Emerging EEG Devices,” Frontiers in Physiology, vol. 9, p. 98, 2018.
[26] T. Chabin, D. Gabriel, E. Haffen, T. Moulin and L. Pazart, “Are the new mobile wireless EEG headsets reliable for the evaluation of musical pleasure?,” PLOS ONE, vol. 15, no. 12, p. e0244820, 2020.
[27] T. Radüntz and B. Meffert, “User Experience of 7 Mobile Electroencephalography Devices: Comparative Study,” JMIR Mhealth Uhealth, vol. 7, no. 9, p. e14474, 2019.
[28] W. Zheng, K. A. Service, J. J. Aftreth, P. H. Sessoms and B. D. Cox, “Validating Mobile Electroencephalographic Systems for Integration into the PhyCORE and Application in Clinical Settings,” Naval Health Research Center, San Diego, 2016.
[29] E. Wascher, H. Heppner, S. O. Kobald, S. Arnau, S. Getzmann and T. Möckel, “Age-Sensitive Effects of Enduring Work with Alternating Cognitive and Physical Load. A Study Applying Mobile EEG in a Real Life Working Scenario,” Frontiers in Human Neuroscience, vol. 9, p. 711, 2016.
[30] D. Hö11e, J. Meekes and M. G. Bleichner, “Mobile ear-EEG to study auditory attention in everyday life,” Behavior Research Methods, 2021.
[31] J. E. M. Scanlon, K. A. Townsend, D. L. Cormier, J. W. P. Kuziek and K. E. Mathewson, “Taking off the training wheels: Measuring auditory P3 during outdoor cycling using an active wet EEG system,” Brain Research, vol. 1716, pp. 50-61, 2019.
[32] R. Zink, B. Hunyadi, S. Van Huffel and M. De Vos, “Mobile EEG on the bike: Disentangling attentional and physical contributions to auditory attention tasks,” Journal of Neural Engineering, vol. 13, no. 4, p. 046017, 2016.
[33] S. Debener, F. Minow, R. Emkes, K. Gandras and M. de Vos, “How about taking a low-cost, small, and wireless EEG for a walk?,” Psychophysiology, vol. 49, no. 11, pp. 1617-21, 2012.
[34] M. De Vos, K. Gandras and S. Debener, “Towards a truly mobile auditory brain-computer interface: Exploring the P300 to take away,” International Journal of Psychophysiology, vol. 91, no. 1, pp. 46-53, 2013.
[35] P. Aspinall, P. Mavros, R. Coyne and J. Roe, “The urban brain: analysing outdoor physical activity with mobile EEG,” British Journal of Sports Medicine, vol. 49, pp. 272-6, 2015.
[36] W. Lin, M. Jiang, J. Tao, Z. Liu, X. Zhang, L. Wu, S. Xu, Y. Kang and Q. Zeng, “Sitting or Walking? Analyzing the Neural Emotional Indicators of Urban Green Space Behavior with Mobile EEG,” Journal of Urban health, vol. 97, no. 2, pp. 191-203, 2020.
[37] F. Dehais, A. Duprés, S. Blum, N. Drougard, S. Scannella, R. N. Roy and F. Lotte, “Monitoring Pilot's Mental Workload Using ERPs and Spectral Power with a Six-Dry-Electrode EEG System in Real Flight Conditions,” Sensors (Basel), vol. 19, no. 6, p. 1324, 2019.
[38] J. J. Norton, D. S. Lee, J. W. Lee, W. Lee, O. Kwon, P. Won, S.-Y. Jung, H. Cheng, J.-W. Jeong, A. Akce, S. Umunna, I. Na, Y. H. Kwon, X.-Q. Wang, Z. Liu, U. Paik, Y. Huang, T. Bretl, W.-H. Yeo and J. A. Rogers, “Soft, curved electrode systems capable of integration on the auricle as a persistent brain-computer interface,” Proceedings of the National Academy of Sciences of the United States of America, vol. 112, no. 13, pp. 3920-5, 2015.
[39] D.-H. Kim, N. Lu, R. Ma, Y.-S. Kim, R.-H. Kim, S. Wang, J. Wu, S. M. Won, H. Tao, A. Islam, K. J. Yu, T.-I. Kim, R. Chowdhury, M. Ying, L. Xu, M. Li, H.-J. Chung, H. Keum, M. McCormick, P. Liu, Y.-W. Zhang, F. G. Omenetto, Y. Huang, T. Coleman and J. A. Rogers, “Epidermal Electronics,” Science, vol. 333, no. 6044, pp. 838-43, 2011.

1. A method of determining behavioral activity of an individual, comprising:
receiving electrical signals pertaining to electrophysiological biopotentials sensed from the skin over a plurality of identified channels;
processing said electrical signals to provide for each channel a likelihood for local muscle activation; and
analyzing said likelihoods based on said channel identification to determine behavioral activity of the individual.

2. The method according to claim 1, wherein said receiving comprises receiving said signals from a set of electrodes placed on a skin of the individual, wherein each channel corresponds to one of said electrodes.

3. The method according to claim 2, wherein said receiving said signals from said set of electrodes is while the individual is performing an unconstrained behavioral activity.

4. The method according to claim 1, wherein said processing comprises generating a square wave signal identifying onsets and cessations of electromyographic events.

5. (canceled)

6. The method according to claim 4, wherein said processing comprises applying an energy tracking operator followed by a rectification operator to said signal.

7. (canceled)

8. The method according to claim 4, wherein said analyzing comprises thresholding amplitudes of said square wave signal.

9. (canceled)

10. The method according to claim 4, wherein said analyzing comprises thresholding times between bouts of said square wave signal.

11. (canceled)

12. The method according to claim 1, comprising filtering said electrical signals, prior to said processing, at frequencies specific to said behavioral activity.

13-17. (canceled)

18. The method according to claim 1, wherein said behavioral activity comprises at least one of chewing, swallowing, blinking, scratching, and changing posture.

19. (canceled)

20. The method according to claim 18, comprising further analyzing said likelihoods to distinguish between different postures of the individual while performing the same behavioral activity.

21. (canceled)

22. The method according to claim 1, comprising:
receiving additional signals indicative of an additional behavioral activity performed by the individual, wherein said additional signals are other than said electrophysiological biopotentials and said additional behavioral activity is other than said determined behavioral activity; and
determining efficiency at which said additional behavioral activity is performed by the individual based on said determined behavioral activity.

23. (canceled)

24. The method according to claim 22, wherein said additional signals comprise location data, and the method comprises determining said additional behavioral activity based on said location data.

25-29. (canceled)

30. A system for determining behavioral activity of an individual, the system comprising:
a set of electrodes adherable to a skin of an individual for sensing electrophysiological biopotentials from the skin over a plurality of identified channels, each corresponding to one of said electrodes; and
a processor in communication with the electrodes, and having a circuit configured to receive from said electrodes signals pertaining to said biopotentials, to process said signals to provide for each channel a likelihood for local muscle activation, and to analyze said likelihoods based on said channel identification to determine behavioral activity of the individual.

31. The system according to claim 30, wherein said circuit is configured to generate a square wave signal identifying onsets and cessations of electromyographic events.

32. The system according to claim 31, wherein said circuit is configured to apply an energy tracking operator followed by a rectification operator to said signal.

33. The system according to claim 31, wherein said circuit is configured to apply thresholding to amplitudes of said square wave signal.

34. The system according to claim 31, wherein said circuit is configured to apply thresholding to times between bouts of said square wave signal.

35. (canceled)

36. The system according to claim 30, wherein said circuit is configured to filter said signals, prior to said processing, at frequencies specific to said behavioral activity.

37-40. (canceled)

41. The system according to claim 30, wherein said behavioral activity comprises at least one of chewing, swallowing, blinking, scratching, and changing posture.

42-44. (canceled)

45. The system according to claim 30, wherein said processor is in communication with a sensor other than said electrode, and is configured to receive from said sensor additional signals indicative of an additional behavioral activity other than said determined behavioral activity, and to determine efficiency at which said additional behavioral activity is performed by the individual based on said determined behavioral activity.

46-50. (canceled)
